(19) United States
(12) Patent Application Publication
(10) Pub. No.: US 2010/0198037 A1
(43) Pub. Date: Aug. 5, 2010

(54) FEEDBACK SENSOR FOR REAL-TIME MANAGEMENT OF SICKLE CELL DISEASE

(76) Inventor: Steven W. Cole, San Carlos, CA (US)

Correspondence Address: SHAY GLENN LLP, 2755 CAMPUS DRIVE, SUITE 210, SAN MATEO, CA, 94403, US

(21) Appl. No.: 12/697,531

(22) Filed: Feb. 1, 2010

Related U.S. Application Data
(60) Provisional application No. 61/148,736, filed on Jan. 30, 2009.
(60) Provisional application No. 61/281,710, filed on Nov. 20, 2009.

(51) Int. Cl. A61B 5/00

(52) U.S. Cl. 600/368

(57) ABSTRACT

Described herein are devices, systems and methods for real-time (or near real-time) monitoring of red blood cell morphology, and in particular, monitoring of sickling to provide an indication of the risk of a the downstream consequences of sickling, such as the pain crises and associated hypoxic tissue damage which may occur as sickling progresses. The monitors describe herein may be continuous (e.g., sampling the subject continuously while worn or activated), or they may operate at a predetermine or selectable sampling rate. In some variations, the devices described herein are worn or applied to the patient non-invasively or minimally invasively.

### CROSS REFERENCE TO RELATED APPLICATIONS

This patent claims priority to the following U.S. Provisional patent applications: Ser. No. 61/148,736, filed on Jan. 30, 2009, titled “FEEDBACK SENSOR FOR REAL-TIME MANAGEMENT OF SICKLE CELL DISEASE,” and Ser. No. 61/281,710, filed on Nov. 20, 2009, titled “FEEDBACK SENSOR FOR REAL-TIME MANAGEMENT OF SICKLE CELL DISEASE.” Both of these applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are sensors for the real-time assessment of red blood cell (RBC) sickling, methods of making and operating them, and methods of providing therapy to patients at risk for sickle cell anemia using them.

### BACKGROUND OF THE INVENTION

Sickle cell disease (SCD) is syndrome of specific health ailments that are caused by a genetic variation in the structure of genes encoding hemoglobin molecules. Hemoglobin is the component of red blood cells (RBCs, or erythrocytes) that carries oxygen from the lungs to body tissues. In SCD, genetically-induced alterations in hemoglobin structure may cause the aberrant hemoglobin protein to polymerize into long chains that distort the shape of RBCs. Normal RBCs efficiently travel through small blood vessels (microvasculature) because they have a circular, saucer-shaped geometry. SCD-related polymerization of hemoglobin produces irregular, jaggedly-shaped (“sickle”-shaped) RBCs that do not pass easily through microvasculature. RBC sickling may reduce cell flow rates through capillaries, and thereby impair the oxygen supply to tissues (hypoxia). The resulting hypoxia induces pathological cell function (e.g. impaired growth or impaired regeneration) and leads to ischemic tissue damage (e.g., cell death due to hypoxia). SCD-related hypoxia induces several specific disease processes, including slow alterations in tissue growth/regeneration that may be asymptomatic, and “acute vasoocclusive crises” involving the sudden onset of intense pain. Acute pain crises are a major contributor to SCD-related disability and health care utilization.

Sickling of RBCs in patients having a genetic variations in a copy of the hemoglobin gene may be triggered and influenced by patient environment and behavior. RBC sickling is a dynamic process that is influenced in part by the amount of oxygen bound to hemoglobin molecules. Low oxygen levels promote the polymerization of SCD hemoglobin, resulting in decreased oxygen delivery to tissues, SCD-related tissue damage, and increased RBC sickling. Progression of sickling in an individual may change over time, typically on the order of minutes.

SCD pathology is typically “managed” clinically by instructing patients to undertake a variety of behavioral measures aimed at limiting tissue hypoxia and thereby averting the vicious cycle of RBC deoxygenation, RBC sickling, and subsequent tissue hypoxia. These behavioral prevention measures include instructions to avoid intense heat or cold, avoid intensive exercise, avoid dehydration, and avoid high altitudes. These behavioral prevention measures are burdensome and detract from SCD patient quality of life. In addition, they may be generally unnecessary, as SCD patients may not be at immediate risk of RBC sickling, pain crises, and associated hypoxic tissue damage on most occasions. However, in the absence of contemporaneous information about RBC sickling status in one's body, general behavioral alterations are necessary to prevent SCD exacerbation on those rare occasions of heightened risk. A significant advance for the proactive management of SCD could be made by providing patients information about their instantaneous risk of disease exacerbation.

The inventor has herein proposed that monitoring of RBC morphology (sickling) may provide sufficient feedback to allow patients to more effectively manage the disorder, much more effectively than the onerous and often contradictory behavioral modifications currently advised.

No commercial or theoretical product is currently available for effective real-time feedback on RBC sickling. Although the characteristic RBC “sickle” morphology has been known for almost a century, to date real-time monitoring of the progression of cell sicking in a patient has not been described. Numerous technologies have been suggested for monitoring of RBC morphology, including flow cytometery, and other optical techniques such as Raman scattering analysis. See, e.g., US 2006/0281068, U.S. Pat. No. 5,798,827, U.S. Pat. No. 6,630,990, and U.S. Pat. No. 7,075,628. However, none of these techniques is compatible with real-time monitoring of RBC morphology, given the need for rapid and continuous monitoring of active subjects, which requires a compact device that can be readily worn without significantly inhibiting normal activity. The devices and methods described herein may address the problems identified above.

### SUMMARY OF THE INVENTION

The present invention relates to device and methods for real-time monitoring of red blood cell morphology, and in particular, real-time monitoring of sickling to provide an indication of the risk of a the downstream consequences of sickling, such as the pain crises and associated hypoxic tissue damage which may occur as sickling progresses. The monitors describe herein may be continuous (e.g., sampling the subject continuously while worn or activated), or they may operate at a predetermine or selectable sampling rate. In some variations, the devices described herein are worn or applied to the patient non-invasively or minimally invasively. For example, the device may be applied by a patch, clip, etc. In some variations the device may be implanted. The sensor component may communicate directly or via wireless connection with an additional (e.g., analysis) component that may provide feedback, analyze, record, or otherwise manipulate the data on RBC morphology. Also described herein are methods of operating the device.

For example, described herein are small wearable devices that may continually monitor biological indicators of RBC sickling to provide SCD patients with real-time information about the near-future risk of disease exacerbation. This monitoring may provide an indication of the risk level associated with sickling (e.g., over the ensuing few minutes to hours). In some variations the device determines the extent of sickling, for example, using an index of RBC morphology. This SCD biofeedback signal would allow users to modify their behavior quickly and specifically during periods of elevated disease risk. For example, exacerbation risk can be gauged by measurements of RBC geometry (e.g., via assessment of saucer vs. jagged morphology via incident light side-scatter, as in flow cytometry), rates of RBC flow-through capillary beds (e.g., via assessment of intervals between consecutive red blood cells' alignments), RBC oxygenation (e.g., via electro-optical or electrochemical detection, as in pulse oximetry), and/or local tissue oxygenation (e.g., via detection of biochemical or protein indicators such as Hypoxia-Inducible Factors/HIFs, possibly by antibody-mediated immunosorbent assays or surface plasmon resonance imaging).

As mentioned above, in some variations, the device (or sensor portion of the device) is non-invasive or minimally invasive. For example, the sensor(s) may be directed to a readily accessible capillary bed such as that found in the earlobe (e.g., by clipping, taping, or otherwise connecting the device to the outside of the earlobe). Other minimally invasive sensors may be attached to the subject's skin via a dermal (or transdermal) patch. In some variations the sensor(s) may be held against the skin by an adhesive. In some variations the sensor(s) may be implanted into the patient, including within a sub-dermal region of the body.

In general, the devices are configured for real-time feedback to SCD patients. For example, the device may provide an easily perceived alarm system (e.g., an audible tone, kinesthetic vibration, etc.) indicating enhanced risk (or risk level). Specific implementation strategies are described in detail below. In general, these devices are wearable real-time SCD “alarm systems” that may identify specific periods during which maximal behavioral prevention of SCD exacerbation would be advisable. This information could help alleviate the burden on SCD patient's which otherwise requires constant behavioral management, and might also facilitate clinical and scientific research on SCD (e.g., in studies identifying presently unknown risk factors/situations, assessing impact of other interventions to ameliorate SCD biology, etc.).

In some variations, the devices used to monitor RBC sickling in real time also include an SCD risk detection method (e.g., a method of determining an ‘index’ of rick), and/or a real-time information-delivery system for signaling to the subject either what the current risk level is, or indicating when an enhanced risk is detected. Additional features may allow recording and retrieving of risk information, which may be used for data collection.

Described herein are several embodiments for the determination and detection of SCD risk using one or more sensors. In particular, devices including one or more sensor(s) and related processors for determining relative RBC morphology (e.g., sickled/jagged vs. non-sickled or saucer-shaped cells). The sensors may be optical or electro-optical (e.g., via. light scattering), acoustic and/or electro-acoustic (e.g., via. ultrasound), electric (e.g., via impedance measurement), or the like. Specific examples of sensors are provided herein, although it should be understood that other methodologies may be applied. The examples described herein are provided as illustrations of the types of sensors that may be used to determine in-vivo detection of RBC morphology from a population of cells within a subject's vascular system. In some variations the sensor is used in conjunction with a natural or artificial conduit (e.g., blood vessel or the like). For example, the sensor may be applied to a capillary region.

In one variation, an electro-optical sensor may be used. For example, an electro-optical sensor may be used to assess RBC morphology, e.g., assessing healthy saucer morphology vs. risky jagged morphology by electro-optical detection of light scattering off of cells passing a unidirectional light beam. In one variation, light (e.g., collimated and/or coherent light of a particular wavelength or wavelengths) may be applied and forward- and side-scatter patterns may be analyzed to determine flow and morphology characteristics of cells moving through the light, akin to flow cytometric devices.

In one variation an electro-optical sensor may be used to measure RBC flow rates specifically in microvasculature structures. In this variation, the flow rate may be examined based on the time interval between the appearance of consecutive RBCs at a fixed sensor position (e.g., within a capillary). As cell sickling increases, the flow rate through narrow vessels (e.g., capillaries) is expected to decrease. In variations of the electro-optical sensors described herein, the sensor may be applied to the skin, which applies the energy to the surface of the skin and detects changes in the shape or motion of the RBCs transdermally (through the skin); optical signals may be subtracted, or optical interference may be used to remove intervening “stationary” signals to distinguish the population of moving RBCs and other blood or lymph cells.

In some variations, RBC oxygenation levels in vascular beds may be detected. For example, colorometric or electrical charge/density analysis of cells passing a fixed peri-vascular sensor may be used to determine oxygenation levels. This information may be used in combination with the cell morphology and rate sensor information, or it may be used by itself. In some variations, multiple sensors may be used.

In some variations, direct detection of RBC oxygenation may be determined. For example, a non-morphological assessment of oxygenation based on RBC biochemical properties may be detected, for example, by mass/charge ratios or electromagnetic characteristics.

In some variations, acoustic or electro-acoustic sensor(s) may be used to determine the motion and/or morphology of RBCs in one or more regions of the body.

In some variations, a cellular physiologic responses to hypoxia in non-RBC cells may be determined by one or more sensors of protein/nucleic acid levels. For example, a sensor (akin to current “gene chip” technology may determine activation of HIF transcription factors in extravascular cells or vascular endothelial cells. Activation or expression of HIP transcription factors may be detected by antibody-linked nanosensors, or by detection of transcription of hypoxia-inducible genes such as Vascular Endothelial Growth Factor detected by nucleic acid complementation, for example. Microarray technologies capable of such detection are available, and may be adapted for use herein, including for use as an additional sensor/modality.

In some variations, a method of risk detection may include correlation of an individual sensor signal(s) with the subsequent development of clinical symptoms, or with more invasive “criterion” measures of RBC sickling or cellular hypoxia. The correlation may be determined from a population (e.g., combining data from multiple subjects) or on an individualized basis (specific to each user), or both. For example, direct biochemical or morphological analyses of cells ex vivo, e.g., by mass spectrometry or flow cytometry, may be used to correlate to sensor output once the sensor has be positioned on a typical or specific subject.

Delivery of forecast SCD exacerbation risk information to patients could be implemented through a variety of optical, aural, or tactile signals. For example, a tone could be sounded during periods of high risk. Alternatively, a series of tones that vary in frequency or volume might be employed to provide graduated information about the relative magnitude of risk. A variant of this approach might employ inaudible vibrations delivered to the skin in the area of sensor placement (e.g., as in the “silent”/“vibrate” ring of current cellular telephones). Regardless of the specific feedback modality, the relationship between feedback signal intensity and measured RBC/oxygenation parameters would be optimized using standard signal detection methodologies (e.g., Receiver Operating Characteristics to provide suitable sensitivity, specificity, and diagnosticity).

The proposed feedback device typically allows a set of behavioral alterations that can be readily undertaken by the subject to prevent further sub-clinical exacerbation and reduce the likelihood of a clinically significant event such as an acute pain crisis. Such instructions (e.g., to normalize body temperature, reduce oxygen expenditure, increase oxygen supply, etc.) may be delivered as part of a training package accompanying the device. The device itself might provide prompts to assist the patient in recalling or using preventive/protective procedures during periods of disease exacerbation. This might be accomplished by aural signaling (e.g., recorded instructions played at an audible volume), or by other coded signals (e.g., a pattern of tactile stimuli that serve as mnemonics to cue previously trained verbal instructions such as 3 acute pulses to recall “temperature, oxygen, rest”).

In some variations the device includes a memory to store accumulating sensor data over time, and an input/output section to support transfer of data to other devices for analysis.

Also described herein are devices including a sensor component (“sensor”) with one or more sensors for detecting RBC sickling and/or associated data from the subject, and a processing component (“processor”) used with sensor. Other sub-components of the system may also be included. The sensor component and the processor component may be coupled togeheter directly or remotely, or they may be integrated together. In some variations the sensor component is applied directly (or implanted) to the subject for longer-term use, while the processor may be carried or worn separately. Thus, the remote receiver may receive sensor data, including “raw” or unprocessed data and/or processed data. The remote receiver may also send information or instructions to the sensor module. In some variations the two wirelessly communicate.

### BRIEF DESCRIPTION OF THE DRAWINGS

FIG. 1 is a schematic of one variation of a system for determining the risk of RBC sickling, pain crises, and associated hypoxic tissue damage as described herein.

### DETAILED DESCRIPTION OF THE INVENTION

In general, the devices and systems described herein are for monitoring in real-time or near-real time the risk of pain crisis due to red blood cell (RBC) sickling and/or any associated risk of hypoxic tissue damage. Also described herein are methods for determining the risk of pain crisis and/or hypoxic tissue damage.

For example, the devices may be wearable devices for determining the ongoing risk of red blood cells sickling, pain crises, and associated hypoxic tissue damage. Such devices may include: a wearable sensor for detecting the morphology of red blood cells; a processor for receiving information from the wearable sensor and assessing the extent of red blood cell sickling in real time; and an output coupled to the processor configured to warn of an elevated risk of pain crises and/or associated hypoxic tissue damage.

As used herein “near real time” and “real time” typically refers to the actual time or approximately (e.g., within 10 seconds, within 20 seconds, within 30 seconds, within 1 minute, within 2 minutes, within 5 minutes or less) time an event occurs. Thus, a device that determines the ongoing risk of RBC sickling, pain crisis and/or hypoxic tissue damage in real time operates by providing an assessment (and possibly an output) within this time frame based on a rapid assessment of cell morphology, for example. This may allow the device to provide rapid, useful feedback to the subject.

Any appropriate sensor may be used, particularly optical and/or acoustic sensors. For example, an optical or photoacoustic sensor (such as those described in US2009/0156932 to Zharov, incorporated herein in its entirety), may be configured for the real-time sensing of blood cell morphology. The processor may incorporate the data received by the sensor/sensing element to determine the extent of sicking by indexing the irregularity in morphology of moving (e.g., blood cells) passing the sensor over time. The processor may be continuous, or it may be activated for a window of time.

The devices may be implantable devices for determining the ongoing risk of red blood cell sickling, pain crises, and associated hypoxic tissue damage. For example, an implantable device may include: an implantable sensor for determining the morphology of red blood cells; a processor for receiving information from the sensor and assessing the extend to red blood cell sickling in real time; and an output coupled to the processor configured to warn of an elevated risk of pain crisis and/or associated hypoxic tissue damage.

Again, any appropriate sensor may be used. In some variations, the sensor is configured to optically scan moving blood cells to determine their morphology. For example, the blood cells may be scanned similar to the techniques used by classical flow cytometric techiniques. Non-invasive variations of these device may also be used.

Methods of determining risk of RBC sickling, pain crises, and associated hypoxic tissue damage in real-time may include the steps of: determining the extent of sickling of red blood cells in real- or near-real time by examining the morphology of the red blood cells; assessing the risk of pain crisis and/or associated hypoxic tissue damage based on the extent of sickling determined; and providing a warning of pain crisis and/or associated hypoxic tissue damage. The method may also include the step of connecting a sensor to a subject, wherein the sensor is configured to determine the morphology of red blood cells in real or near-real time.

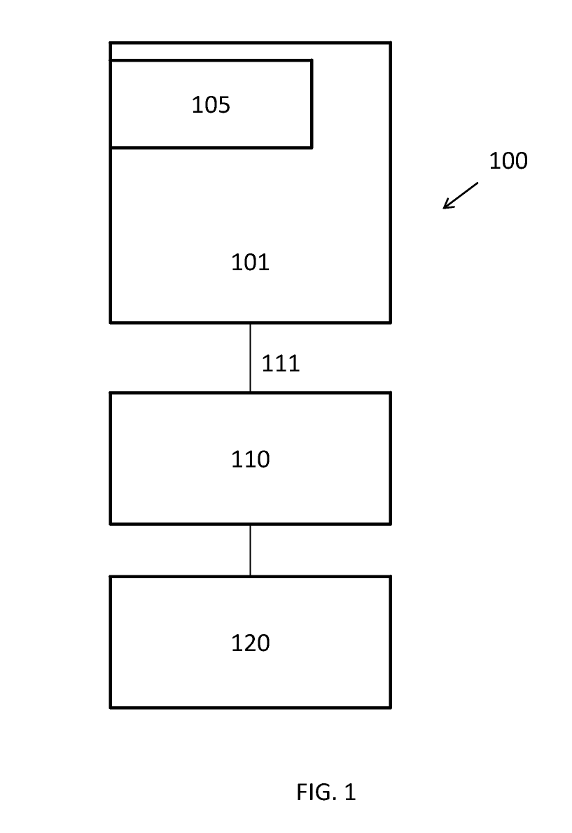

FIG. 1 illustrates one variation of a system for determining the risk of RBC sickling, pain crises, and associated hypoxic tissue damage. In this example, the system includes: a sensing element (101) comprising a sensor (105) for detecting the morphology of red blood cells in real time or near-real time, wherein the sensing element is configured to contact a subject (not shown); a processing element (110) may be in communication with the sensing element (101) directly (111) or wirelessly. The processing element (110) is typically configured for receiving information from the sensing element (101) and/or sensor (101) and assessing the extent of red blood cell sickling in real time. An output element (120) is in communication (including wirelessly) with the processing element (110) and is configured to warn of an elevated risk of pain crises and/or associated hypoxic tissue damage. The output may be visual (e.g., including lights, display, or the like), aural (e.g., bell, alarm, recorded voice, etc.), tactile (e.g., vibration, etc.) and/or any other alert means.

Although the foregoing inventions have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A wearable device for determining the ongoing risk of red blood cells sickling, pain crises, and associated hypoxic tissue damage, the device comprising:
a wearable sensor for detecting the morphology of red blood cells;
a processor for receiving information from the wearable sensor and assessing the extent of red blood cell sickling in real time; and
an output coupled to the processor configured to warn of an elevated risk of pain crises and/or associated hypoxic tissue damage.

2. An implantable device for determining the ongoing risk of red blood cell sickling, pain crises, and associated hypoxic tissue damage, the device comprising:
an implantable sensor for determining the morphology of red blood cells;
a processor for receiving information from the sensor and assessing the extend to red blood cell sickling in real time; and
an output coupled to the processor configured to warn of an elevated risk of pain crisis and/or associated hypoxic tissue damage.

3. Method of determining risk of RBC sickling, pain crises, and associated hypoxic tissue damage in real-time, the method comprising:
determining the extent of sickling of red blood cells in real- or near-real time by examining the morphology of the red blood cells;
assessing the risk of pain crisis and/or associated hypoxic tissue damage based on the extent of sickling determined; and
providing a warning of pain crisis and/or associated hypoxic tissue damage.

4. The method of claim 3, further comprising the step of connecting a sensor to a subject,
wherein the sensor is configured to determine the morphology of red blood cells in real or near-real time.

5. A system for determining the risk of RBC sickling, pain crises, and associated hypoxic tissue damage, the system comprising:
a sensing element comprising a sensor for detecting the morphology of red blood cells in real time or near-real time, wherein the sensing element is configured to contact a subject;
a processing element in communication with the sensing element, wherein the processing element is configured for receiving information from the sensor and assessing the extent of red blood cell sickling in real time; and
an output element in communication with the processing element configured to warn of an elevated risk of pain crises and/or associated hypoxic tissue damage.

6. The system of claim 5, wherein the sensing element wirelessly communicates with the processing element.
